SEQUENCE LISTING (18 sequences)

SEQ ID NO: 1 - protein, 100 aa, Artificial Sequence
  Beta-CTF domain
MDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 100 aa, Artificial Sequence
  Beta-CTF domain
MDAEFRHDSGYEVHHQKLVFFAEDVGSNKGAIIGLMVGGVVIATVVVITLVMLKKKQYTSIHHGVVEVDAAVTPEERHLSKMQQNGYENPTYKFFEQMQN

SEQ ID NO: 3 - protein, 100 aa, Artificial Sequence
  Beta-CTF domain
MDAEFRHDSGYEVHHQKLVFFAEDVGSNKGAIIGLMVGGVVIATVIIITLVMLKKKQYTSIHHGVVEVDAAVTPEERHLSKMQQNGYENPTYKFFEQMQN

SEQ ID NO: 4 - protein, 100 aa, Artificial Sequence
  Beta-CTF domain
MDAEFRHDSGYEVHHQKLVFFAEDVGSNKGAIIGLMVGGVVIATVIGITLVMLKKKQYTSIHHGVVEVDAAVTPEERHLSKMQQNGYENPTYKFFEQMQN

SEQ ID NO: 5 - protein, 100 aa, Artificial Sequence
  Beta-CTF domain
MDAEFRHDSGYEVHHQKLVFFAEDVGSNKGAIIGLMVGGVVIATVIFITLVMLKKKQYTSIHHGVVEVDAAVTPEERHLSKMQQNGYENPTYKFFEQMQN

SEQ ID NO: 6 - protein, 100 aa, Artificial Sequence
  Beta-CTF domain
MDAEFRHDSGYEVHHQKLVFFGEDVGSNKGAIIGLMVGGVVIATVIVITLVMLKKKQYTSIHHGVVEVDAAVTPEERHLSKMQQNGYENPTYKFFEQMQN

SEQ ID NO: 7 - protein, 100 aa, Artificial Sequence
  Beta-CTF domain
MDAEFRHDSGYEVHHQKLVFFAQDVGSNKGAIIGLMVGGVVIATVIVITLVMLKKKQYTSIHHGVVEVDAAVTPEERHLSKMQQNGYENPTYKFFEQMQN

SEQ ID NO: 8 - protein, 8 aa, Artificial Sequence
  Hydrophilic moiety
DYKDDDDK

SEQ ID NO: 9 - protein, 108 aa, Artificial Sequence
  Gamma-secretase substrate
MDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQNDYKDDDDK

SEQ ID NO: 10 - protein, 108 aa, Artificial Sequence
  Gamma-secretase substrate
MDAEFRHDSGYEVHHQKLVFFAEDVGSNKGAIIGLMVGGVVIATVVVITLVMLKKKQYTSIHHGVVEVDAAVTPEERHLSKMQQNGYENPTYKFFEQMQNDYKDDDDK

SEQ ID NO: 11 - protein, 108 aa, Artificial Sequence
  Gamma-secretase substrate
MDAEFRHDSGYEVHHQKLVFFAEDVGSNKGAIIGLMVGGVVIATVIIITLVMLKKKQYTSIHHGVVEVDAAVTPEERHLSKMQQNGYENPTYKFFEQMQNDYKDDDDK

SEQ ID NO: 12 - protein, 108 aa, Artificial Sequence
  Gamma-secretase substrate
MDAEFRHDSGYEVHHQKLVFFAEDVGSNKGAIIGLMVGGVVIATVIGITLVMLKKKQYTSIHHGVVEVDAAVTPEERHLSKMQQNGYENPTYKFFEQMQNDYKDDDDK

SEQ ID NO: 13 - protein, 108 aa, Artificial Sequence
  Gamma-secretase substrate
MDAEFRHDSGYEVHHQKLVFFAEDVGSNKGAIIGLMVGGVVIATVIFITLVMLKKKQYTSIHHGVVEVDAAVTPEERHLSKMQQNGYENPTYKFFEQMQNDYKDDDDK

SEQ ID NO: 14 - protein, 108 aa, Artificial Sequence
  Gamma-secretase substrate
MDAEFRHDSGYEVHHQKLVFFGEDVGSNKGAIIGLMVGGVVIATVIVITLVMLKKKQYTSIHHGVVEVDAAVTPEERHLSKMQQNGYENPTYKFFEQMQNDYKDDDDK

SEQ ID NO: 15 - protein, 108 aa, Artificial Sequence
  Gamma-secretase substrate
MDAEFRHDSGYEVHHQKLVFFAQDVGSNKGAIIGLMVGGVVIATVIVITLVMLKKKQYTSIHHGVVEVDAAVTPEERHLSKMQQNGYENPTYKFFEQMQNDYKDDDDK

SEQ ID NO: 16 - DNA, 32 bp, Artificial Sequence
  Primer
ggaattccatatggatgcagaattccgacatg

SEQ ID NO: 17 - DNA, 61 bp, Artificial Sequence
  Primer
cgcggatccctatttatcgtcatcgtctttgtagtcgttctgcatctgctcaaagaacttg

SEQ ID NO: 18 - DNA, 327 bp, Artificial Sequence
  DNA Encoding SEQ. ID. NO. 9
atggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaacgactacaaagacgatgacgataaatag

(19) United States
(12) Patent Application Publication
(10) Pub. No.: US 2006/0036077 A1
(43) Pub. Date: Feb. 16, 2006

(54) Gamma secretase substrates and in vitro assays

(76) Inventors: Yueming Li, New York, NY (US); Min Xu, Ambler, PA (US); Qian Huang, Orefield, PA (US); Stephen J. Gardell, Woodbridge, CT (US)

Correspondence Address: MERCK AND CO., INC, P O BOX 2000, RAHWAY, NJ, 07065-0907, US

(21) Appl. No.: 11/243,578

(22) Filed: Oct. 5, 2005

Related U.S. Application Data
(63) Continuation of application No. 10/275,025, filed on Jan. 17, 2003.

(51) Int. Cl. C07K 14/47; C12Q 1/37

(52) U.S. Cl. 530/350; 435/23

(57) ABSTRACT

The present invention features γ-secretase substrates and in vitro assays for measuring γ-secretase activity employing such substrates. The γ-secretase substrates described herein contain a hydrophilic polypeptide moiety covalently joined to the carboxyl terminus of a β-CTF domain. A “β-CTF domain” is a polypeptide that can be cleaved by γ-secretase and which approximates the C-terminal fragment (amino acids 596-695) of APP produced after cleavage of APP by a β-secretase, or is a functional derivative thereof.

### CROSS-REFERENCE TO RELATED APPLICATIONS

The present application claims priority to provisional application U.S. Ser. No. 60/201,053, filed May 1, 2000, hereby incorporated by reference herein.

### BACKGROUND OF THE INVENTION

The references cited herein are not admitted to be prior art to the claimed invention.

Amyloid precursor protein (APP) is a ubiquitous membrane-spanning (type 1) glycoprotein that undergoes a variety of proteolytic processing events. (Selkoe, 1998. Trends in Cell Biology 8, 447-453.) Sequential cleavage of APP by the β- and γ-secretases generate the N- and C-termini, respectively, of Aβ peptides which comprise amyloid plaques in the brain parenchyma of patients with Alzheimer's disease. APP proteolysis by α-secretase occurs within the Aβ peptide domain thereby precluding formation of the amyloidogenic peptides. The C-termini of the Aβ peptides are heterogeneous. Peptides of 40 or 42 amino acids in length (Aβ40 and Aβ42, respectively) are typically generated. Aβ42 is more prone to aggregation than Aβ40 and is the major component of amyloid plaque. (Jarrett et al., 1993. Biochemistry 32, 4693-4697; and Kuo et al., 1996. J. Biol. Chem. 271, 4077-4081.)

The scissile bond for cleavage of APP by γ-secretase appears to be situated within a transmembrane domain. It is unclear as to whether the C-termini of Aβ40 and Aβ42 are generated by a single protease with sloppy specificity or two distinct proteases. Recent studies suggest that γ-secretase also cleaves within the transmembrane region of Notch thereby releasing the Notch intracellular domain which controls crucial cell fate decisions during development. (De Strooper et al., 1999. Nature 398, 518-522.)

### SUMMARY OF THE INVENTION

The present invention features y-secretase substrates and in vitro assays for measuring γ-secretase activity employing such substrates. The γ-secretase substrates described herein contain a hydrophilic polypeptide moiety covalently joined to the carboxyl terminus of a β-CTF domain.

A “β-CTF domain” is a polypeptide that can be cleaved by γ-secretase and which approximates the C-terminal fragment (amino acids 596-695) of APP produced after cleavage of APP by a β-secretase, or is a functional derivative thereof. Preferably, the β-CTF domain can be cleaved by a γ-secretase to produce a fragment between about 39 to about 43 amino acids in length. The preferred size ranges takes into account the generation of peptides Aβ40 and Aβ42 from naturally occurring APP by the sequential actions of β-secretase and γ-secretase.

The hydrophilic polypeptide moiety is preferably chosen to increase the solubility of the γ-secretase substrate in a zwitterionic detergent. Hydrophilic moieties can be obtained taking into account the known charges and polarity of different amino acid R groups.

Thus, a first aspect of the present invention features a γ-secretase substrate. The γ-secretase substrate contains a hydrophilic polypeptide moiety covalently joined to the carboxyl terminus of a β-CTF domain.

Another aspect of the present invention describes a nucleic acid comprising a nucleotide base sequence encoding a γ-secretase substrate. Preferably, the nucleic acid is an expression vector.

Another aspect of the present invention describes a recombinant cell comprising a nucleic acid encoding a γ-secretase substrate.

Another aspect of the present invention describes a method for assaying γ-secretase activity comprising the use of an effective amount of a zwitterionic detergent and a γ-secretase substrate. γ-Secretase activity can be obtained from cells producing γ-secretase in a solubilized form or in a membrane-bound form. The effective amount of a zwitterionic detergent is a concentration of zwitterionic detergent where γ-secretase produces detectable cleavage of the γ-secretase substrate.

The method can be performed by measuring product formation resulting from γ-secretase substrate cleavage. Measuring can be performed by qualitative or quantitative techniques.

Another aspect of the present invention describes a method for measuring the ability of a compound to affect γ-secretase activity comprising the steps of: (a) combining together a γ-secretase substrate, a compound, and a preparation comprising γ-secretase activity, under reaction conditions allowing for γ-secretase activity, and (b) measuring γ-secretase activity. The reaction conditions allowing for γ-secretase activity comprise an effective amount of a zwitterionic detergent.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 provides a schematic representation of a fusion protein consisting sequentially of an N-terminal Met (M), APP597-695 and the Flag tag (Flag) sequence, and its processing by γ-secretase. The Aβ40- and Aβ42-related products (M-Aβ40 and M-Aβ42, respectively) are detected by electrochemiluminescence (ECL) using biotinylated 4G8 antibody and ruthenylated G2-10 or FCA3542, respectively.

FIGS. 2A-2D illustrate results of experiments characterizing detergent-solubilized γ-secretase activity. (A) Dependence of M-Aβ40 formation on the substrate concentration. The data show the ECL signal after a 90 minute incubation at 37° C. (B) Time dependence of M-Aβ40 formation by “solubilized γ-secretase”. The ECL signals are shown. (C) pH dependence of “solubilized γ-secretase” activity scoring for generation of the M-Aβ40. The ECL signals are shown. (D) Inhibition of in vitro γ-secretase activity by pepstatin. The impact of pepstatin on the generation of M-Aβ40 (∘) and M-Aβ42 (●) is shown. The ECL data is expressed as % of activity observed in the absence of pepstatin. FIGS. 2A, 2B and 2C show the mean values from two independent experiments. FIG. 2D shows the mean±SD (n=5).

### DETAILED DESCRIPTION OF THE INVENTION

The present invention features γ-secretase substrates and assays for detecting γ-secretase activity employing such substrates. The γ-secretase substrate can be cleaved by γ-secretase activity.

Assaying for γ-secretase activity can be used, for example, to purify the enzyme, to characterize the enzyme, to screen for compounds able to modulate γ-secretase activity, and to test the ability of a particular compound to affect γ-secretase activity. Examples of compounds able to modulate γ-secretase activity include γ-secretase inhibitors. Inhibitors can be employed for different purposes, such as in the treatment of Alzheimer's disease or characterization of the biological importance of γ-secretase.

γ-Secretase Substrate

The γ-secretase substrate is a fusion protein comprising a β-CTF domain and a hydrophilic polypeptide moiety. The β-CTF domain provides a polypeptide that can be cleaved by γ-secretase activity. The hydrophilic polypeptide moiety allows for the β-CTF domain to be cleaved by detergent-solubilized γ-secretase by promoting substrate solubility.

The β-CTF domain approximates the C-terminal fragment of APP after cleavage by β-secretase or is a functional derivative thereof. By approximating the β-CTF portion of APP the γ-secretase substrate takes into account the cleavage of APP by α- or β-secretase appearing to be a prerequisite for γ-secretase-mediated processing.

A “functional derivative thereof” has a sufficient sequence similarity to the β-CTF portion of APP such that it can be cleaved by γ-secretase. Examples of modifications to a β-CTF portion of APP to produce a functional derivative include additions, deletions, and substitutions. The effect of a particular modification can be measured using reaction conditions described herein that allow for γ-secretase cleavage of a γ-secretase substrate. Preferred modifications do not cause a substantial decrease in activity. Preferably, additions and deletions if present are located at the 5′ or 3′ end rather than being internal.

A “substantial decrease in activity” occurs when the observed activity of γ-secretase is decreased 10 fold or more compared to activity observed using a SEQ. ID. NO. 9 substrate incubated with cell membranes or detergent-solubilized γ-secretase in the presence of 0.25% CHAPSO in buffer B (50 mM PIPES, pH 7.0, 5 mM MgCl₂, 5 mM CaCl₂, 150 mM KCl) at 37° C. (described in Example 4, infra.). In different embodiments there is less than at a 5 fold or 2 fold decrease in activity.

Substitutions in the substrate not causing a substantial decrease in activity can be initially designed taking into account differences in naturally occurring amino acid R groups. An R group affects different properties of the amino acid such as physical size, charge and hydrophobicity. Amino acids can be divided into different groups as follows: neutral and hydrophobic (alanine, valine, leucine, isoleucine, proline, tryptophan, phenylalanine, and methionine); neutral and polar (glycine, serine, threonine, tyrosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acids groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain. (See, Ausubel, Current Protocols in Molecular Biology, John Wiley, 1987-1998, Supplement 33 Appendix 1C.)

SEQ. ID. NO. 1 provides an example of a β-CTF domain. SEQ. ID. NO. 1 is a naturally occurring sequence corresponding to the β-CTF portion of APP (amino acids 596-695) along with an N-terminus methionine. The N-terminus methionine facilitates recombinant production of the substrate. SEQ. ID. NO. 1 is as follows:

SEQ. ID. NO.1

MDAEFRHDSGYEVHHQKLVFFAEDVGSNKGAIIGLMVGGVVIATV
IVITLVMLKKKQYTSIHHGVVEVDAAVTPEERHLSKMQQNGYENP
TYKFFEQMQN.

Derivatives of the β-CTF portion of APP provided in SEQ. ID. NO. 1 that are able to be cleaved in vivo by γ-secretase are well known in the art. (See, for example, Lichtenthaler et al., 1997. Biochemistry 36, 15396-15403; and Selkoe, 1999. Nature 399:A23-A31, which is not admitted to be prior art to the claimed invention.) Such derivatives can themselves provide a β-CTF domain or can serve as a starting point for creating additional derivatives.

Examples of naturally occurring derivatives of SEQ. ID. NO. 1 are provided by SEQ. ID. NOs. 2-7. SEQ. ID. NOs. 2-7 correspond to the β-CTF portion of APP (amino acids 596-695) and also contain an N-terminus methionine. SEQ. ID. NOs. 2-7, where differences between these sequences and SEQ. ID. NO. 1 are highlighted, are provided as follows:

SEQ. ID. NO.2

MDAEFRHDSGYEVHHQKLVFFAEDVGSNKGAIIGLMVGGVVIAT
VVVITLVMLKKKQYTSIHHGVVEVDAAVTPEERHLSKMQQNGYE
NPTYKFFEQMQN;

SEQ. ID. NO.3

MDAEFRHDSGYEVHHQKLVFFAEDVGSNKGAIIGLMVGGVVIAT
VIIITLVMLKKKQYTSIHHGVVEVDAAVTPEERHLSKMQQNGYE
NPTYKFFEQMQN;

SEQ. ID. NO.4

MDAEFRHDSGYEVHHQKLVFFAEDVGSNKGAIIGLMVGGVVIAT
VIGITLVMLKKKQYTSIHHGVVEVDAAVTPEERHLSKMQQNGYE
NPTYKFFEQMQN;

SEQ. ID. NO.5

MDAEFRHDSGYEVHHQKLVFFAEDVGSNKGAIIGLMVGGVVIAT
VIFITLVMLKKKQYTSIHHGVVEVDAAVTPEERHLSKMQQNGYE
NPTYKFFEQMQN;

SEQ. ID. NO.6

MDAEFRHDSGYEVHHQKLVFFGEDVGSNKGAIIGLMVGGVVIAT
VIVITLVMLKKKQYTSIHHGVVEVDAAVTPEERHLSKMQQNGYE
NPTYKFFEQMQN;

SEQ. ID. NO.7

MDAEFRHDSGYEVHHQKLVFFAQDVGSNKGAIIGLMVGGVVIAT
VIVITLVMLKKKQYTSIHHGVVEVDAAVTPEERHLSKMQQNGYE
NPTYKFFEQMQN;

In an embodiment of the present invention, the β-CTF domain sequence comprises, consists essentially of, or consists of, a polypeptide substantially similar to SEQ. ID. NO. 1. Preferably, the β-CTF domain sequence comprises, consists essentially of, or consists of, a sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7.

“Substantially similar” indicates a sequence similarity of at least about 80% to a reference sequence. In different embodiments the sequence similarity is at least about 90%, at least about 95% or 100%. Sequence similarity can be determined using techniques well known in the art, such as those described by Altschul et al., 1997. Nucleic Acids Res. 25, 3389-3402, hereby incorporated by reference herein. In one embodiment sequence similarity is determined using tBLASTn search program with the following parameters: MATRIX:BLOSUM62, PER RESIDUE GAP COST: 11, and Lambda ratio: 1.

“Consists essentially” indicates that the reference sequence can be modified by N-terminal and/or C-terminal additions or deletions that do not cause a substantial decrease in the ability of the γ-secretase substrate to be cleaved compared to the reference sequence. Preferably, additions or deletions if present are less than 5 amino acids on either end. An example of a deletion is the removal of the N-terminal methionine.

A substantial decrease in the ability of the γ-secretase substrate to be cleaved is a decrease of 10 fold or more compared to activity observed using a reference substrate incubated with cell membranes or detergent-solubilized γ-secretase in the presence of 0.25% CHAPSO in buffer B (50 mM PIPES, pH 7.0, 5 mM MgCl₂, 5 mM CaCl₂, 150 mM KCl) at 37° C. (described in Example 4, infra.). In different embodiments there is less than a 5 fold or 2 fold decrease in activity.

The second component of the γ-secretase substrate, the hydrophilic polypeptide moiety, is preferably chosen to increase the solubility of the γ-secretase substrate in a zwitterionic detergent. Hydrophilic moieties can obtained taking into account the known charges and polarity of different amino acid R groups. Preferably, the presence of the hydrophilic moiety does not result in a substrate having a substantial decrease in activity.

Different embodiments concerning the overall length and charge of the hydrophilic moiety are provided as follows: in different embodiments concerning the length, the length is about 5 to about 20 amino acids, about 8 to about 12 amino acids, or about 8 amino acids; in different embodiments concerning the overall charge, the charge is greater than ±2, ±3, or ±4. With respect to a negative charge, a greater charge indicates a higher negative charge value.

Preferably, the hydrophilic moiety comprises, consists essentially of, or consists of, a polypeptide substantially identical to SEQ. ID. NO. 8: DYKDDDDK. Substantially identical to SEQ. ID. NO. 8 indicates that within a corresponding 8 amino acid stretch (no gaps) there is a two, one, or zero amino acid difference. Preferably, the hydrophilic moiety consists of the amino acid sequence of SEQ. ID. NO. 8.

In an embodiment of the present invention concerning the overall structure of the γ-secretase substrate, the γ-secretase substrate comprises, consists essentially of, or consists of, a sequence substantially similar to SEQ. ID. NO. 9. SEQ. ID. NO. 9 corresponds to SEQ. ID. NO. 1 along with a carboxyl terminal SEQ. ID. NO. 8 sequence. Preferably, the γ-secretase substrate comprises, consists essentially of, or consists of, a sequence selected from the group consisting of: SEQ. ID. NO. 9, SEQ. ID. NO. 10, SEQ. ID. NO. 11, SEQ. ID. NO. 12, SEQ. ID. NO. 13, SEQ. ID. NO. 14, and SEQ. ID. NO. 15.

SEQ. ID. NOs. 9-15 are provided as follows (differences between SEQ. ID. NO. 9 and SEQ. ID. NOs. 10-15 are noted in bold and underlined):

SEQ. ID. NO.9

MDAEFRHDSGYEVHHQKLVFFAEDVGSNKGAIIGLMVGGVVIATVIVITL
VMILKKKQYTSIHHGVVEVDAAVTPEERHLSKMQQNGYENPTYKFFEQMQ
NDYKDDDDK;

SEQ. ID. NO.10

MDAEFRHDSGYEVHHQKLVFFAEDVGSNKGAIIGLMVGGVVIATVVVITL
VMILKKKQYTSIHHGVVEVDAAVTPEERHLSKMQQNGYENPTYKFFEQMQ
NDYKDDDDK;

SEQ. ID. NO.11

MDAEFRHDSGYEVHHQKLVFFAEDVGSNKGAIIGLMVGGVVIATVIIITL
VMILKKKQYTSIHHGVVEVDAAVTPEERHLSKMQQNGYENPTYKFFEQMQ
NDYKDDDDK;

SEQ. ID. NO.12

MDAEFRHDSGYEVHHQKLVFFAEDVGSNKGAIIGLMVGGVVIATVIGITL
VMILKKKQYTSIHHGVVEVDAAVTPEERHLSKMQQNGYENPTYKFFEQMQ
NDYKDDDDK;

SEQ. ID. NO.13

MDAEFRHDSGYEVHHQKLVFFAEDVGSNKGAIIGLMVGGVVIATVIFITL
VMILKKKQYTSIHHGVVEVDAAVTPEERHLSKMQQNGYENPTYKFFEQMQ
NDYKDDDDK;

SEQ. ID. NO.14

MDAEFRHDSGYEVHHQKLVFFGEDVGSNKGAIIGLMVGGVVIATVIVITL
VMILKKKQYTSIHHGVVEVDAAVTPEERHLSKMQQNGYENPTYKFFEQMQ
NDYKDDDDK;

Based on the disclosure provided herein γ-secretase substrates can be produced using standard biochemical synthesis and recombinant nucleic acid techniques. Techniques for chemical synthesis of polypeptides are well known in the art. (See, for example, Vincent, in Peptide and Protein Drug Delivery, New York, N.Y., Dekker, 1990.)

Recombinant synthesis techniques for polypeptides are also well known in the art. Such techniques employ a nucleic acid template for polypeptide synthesis. Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. The translation of a particular codon into a particular amino acid is well known in the art (see, e.g., Lewin GENES IV, p. 119, Oxford University Press, 1990).

Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=IIe=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Recombinant synthesis of polypeptides is achieved in a host cell using an expression vector. An expression vector contains recombinant nucleic acid encoding for a desired polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. A preferred element is a polyadenylation signal providing for processing in eukaryotic cells. Other preferred elements include an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Nucleic acid encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

Techniques for introducing nucleic acid into an appropriate environment for expression, for expressing the nucleic acid to produce protein, and for isolating expressed proteins are well known in the art. Examples of such techniques are provided in references such as Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989.

γ-Secretase Assay

The γ-secretase substrate can be employed in assays measuring membrane-bound or detergent-solubilized γ-secretase. Production of cleavage products can be detected by Aβ peptide or hydrophilic moiety product formation.

Solubilized γ-secretase can be obtained from cells producing γ-secretase. Recovery of soluble γ-secretase activity is achieved using a zwitterionic detergent during membrane extraction. Preferably, the amount of zwitterionic detergent is about 1% to about 2%. Examples of cells producing γ-secretase include HeLa S3, human embryonic kidney (HEK293) cells and Chinese hamster ovary (CHO) cells. Examples of zwitterionic detergents include CHAPSO and CHAPS.

Assay conditions employing membrane-bound or detergent-solubilized γ-secretase allow for detectable γ-secretase activity. Such conditions include an effective amount of a zwitterionic detergent, a buffer, and an appropriate temperature.

An effective amount of a particular zwitterionic detergent results in detectable cleavage. Suitable detergents and amounts can be determined by evaluating the effect of a particular detergent on γ-secretase activity. Preferred zwitterionic detergents present during the assay are CHAPS and CHAPSO. A preferred percentage of such detergents is about 0.1% to about 0.5%.

An example of a reaction condition allowing for γ-secretase activity is provided as follows: 1.7 μM substrate incubated with cell membranes or detergent solubilized γ-secretase in the presence of 0.25% CHAPSO in buffer B (50 mM PIPES, pH 7.0, 5 mM MgCl₂, 5 mM CaCl₂, 150 mM KCl) at 37° C. (described in Example 4, infra.). Such conditions can be used as a standard to determine effects of different components. Based on the present disclosure such reaction conditions can be altered to provide a wide range of additional reaction conditions allowing for γ-secretase activity. Preferably, changes to the reaction conditions do not result in a substantial decrease in activity.

γ-Secretase activity can be stopped using techniques well known in the art for stopping enzymatic reactions. Preferably, γ-secretase activity is stopped using reagents compatible with subsequent analysis.

Cleavage of γ-secretase substrates can be measured by detecting formation of an Aβ type product or a product containing the hydrophilic moiety. The presence of either of these products can be measured using techniques such as those employing antibodies and radioactive, electrochemiluminescent or fluorescent labels. If needed or desirable, a purification step enriching the different products may be employed. Examples of purification steps include the use of antibodies, separation gels, and columns.

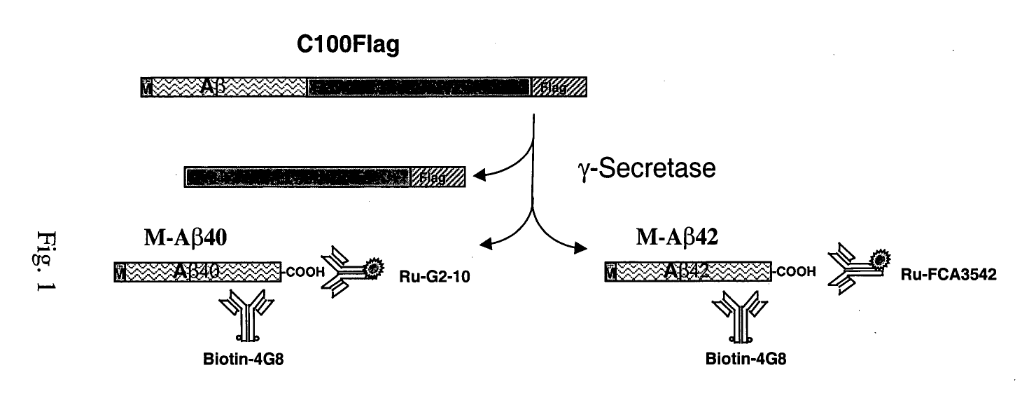

Preferably, cleavage of γ-secretase is assayed for by detecting the presence of Aβ-40 or Aβ-42. FIG. 1 illustrates a preferred method for product detection employing electrochemiluminescence with a capture antibody and an antibody specific for either Aβ-40 or Aβ-42. The capture antibody is used to enrich the products and hence, produce a higher signal.

### EXAMPLES

Examples are provided below to further illustrate different features and advantages of the present invention. The examples also illustrate useful methodology for practicing the invention. These examples do not limit the claimed invention.

### Example 1

### Recombinant Production of the γ-Secretase Substrate

A DNA fragment encoding amino acids 596-695 of the 695 amino acid isoform of APP (APP695) and SEQ. ID. NO. 8 at the C-terminus was generated by PCR amplification of APP695 cDNA using appropriate primers. The employed primers had the following sequences: SEQ. ID. NO. 16: ggaattccatATGGATGCAG AATTCCGACATG; and SEQ. ID. NO. 17: cgcggatccCTAtttatcgtcatcgtctttgtagtcGTT CTGCATCTGCTCAAAGAACTTG.

The Met that serves as the translation start site is residue 596 of APP695 (the P1 residue with respect to the β-secretase cleavage site). This DNA fragment was inserted into the procaryotic expression vector pET2-21b (Novagen, Madison, Wis.). The recombinant protein of SEQ. ID. NO. 9 was overproduced in E. coli [strain BL21(DE3)] and purified by Mono-Q column chromatography (Pharmacia Biotech).

SEQ. ID. NO. 18 provides a nucleic acid sequence encoding for the recombinant protein of SEQ. ID. NO. 9 along with a stop codon.

SEQ. ID. NO. 18

ATGGATGCAGAATTCCGACATGACTCAGGATATGAAGTTCATCATCAAAA
ATTGGTGTTCTTTGCAGAAGATGTGGGTTCAAACAAAGGTGCAATCATTG
GACTCATGGTGGGCGGTGTTGTCATAGCGACAGTGATCGTCATCACCTTG
GTGATGCTGAAGAAGAAACAGTACACATCCATTCATCATGGTGTGGTGGA
GGTTGACGCCGCTGTCACCCCAGAGGAGGCGCCACCTGTCCAAGATGCAG
CAGAACGGCTACGAAAATCCAACCTACAAGTTCTTTGAGCAGATGCAGAA
CgactacaaagacgatgacgataaaTAG

### Example 2

### Aβ Peptide Detection

The Aβ peptides were detected using a sandwich assay employing an antibody to capture the peptide and an antibody to detect the presence of the peptide. Detection was achieved by using ECL (Yang et al., 1994. Bio/Technology 12, 193-194; Khorkova et al., 1998. Journal of Neuroscience Methods 82, 159-166), and an Origen 1.5 Analyzer (Igen Inc., Gaithersburg, Md.).

Capture was performed using the 4G8 murine monoclonal antibody (Senetek PLC, Maryland Heights, Mo.). The 4G8 murine monoclonal antibody binds an epitope in the Aβ peptide (within amino acids 18-21) that is immediately distal to the α-secretase cleavage site. The 4G8 monoclonal antibody was biotinylated with Biotin-LC-Sulfo-NHS-Ester (Igen Inc.).

Detection was achieved using the G2-10 murine monoclonal antibody and the FCA3542 rabbit antibody. The G2-10 murine monoclonal antibody (provided by K. Beyreuther, University of Heidelberg, Germany) binds the C-terminus that is exposed after γ-secretase-mediated cleavage to generate amino acid 40 of the Aβ40 peptide. (Ida et al., 1996. J. Biol. Chem. 271, 22908-22914). The FCA3542 rabbit antibody (provided by F. Checler, IPMC du CNRS, Valbonne, France) binds the C-terminus that is exposed after γ-secretase-mediated cleavage to generate amino acid 42 of the Aβ42 peptide. (Barelli et al., 1997. Molecular Medicine 3, 695-707.)

The G2-10 and FCA3542 antibodies were ruthenylated with TAG-NHS Ester (Igen Inc.). Aβ(x-40) was detected with biotinylated 4G8 and ruthenylated G2-10. Aβ(x-42) was detected with biotinylated 4G8 and ruthenylated FCA3542.

### Example 3

### Membrane Preparation and Detergent Solubilization

HeLa S3 cells from American Type Culture Collection (Rockville, Md.) were grown in bioreactors (Analytical Biological Services; Wilmington, Del.) in 90% DMEM, 10% fetal bovine serum, 2 mM glutamine and 100 μg/ml each of penicillin and streptomycin. Frozen HeLa S3 cells were resuspended in buffer A (50 mM MES, pH 6.0, 5 MM MgCl₂, 5 mM CaCl₂, 150 mM KCl) containing “complete” protease inhibitor cocktail (Boehringer Mannheim, Indianapolis, Ind.). The cells were broken by single-pass through a French Press (Spectronic Instruments, Rochester, N.Y.). Cell debris and nuclei were removed by centrifugation at 800×g for 10 minutes. The supernatant solutions were centrifuged at 100,000×g for 60 minutes. The ensuing pellets were resuspended in buffer A and the centrifugation was repeated. The final membrane pellets were resuspended in buffer A to yield a protein concentration of approximately 12 mg/ml. All procedures were performed at 4° C. The membranes were stored at −70° C.

HeLa cell membranes were treated with varying amounts of CHAPSO (up to 2.0%) followed by centrifugation. The supernatant solutions (solubilized fractions) and the pellets (membrane fractions) were then assayed for γ-secretase activity with the SEQ. ID. NO. 9 substrate.

More γ-secretase activity is recovered in the solubilized fraction, relative to the detergent-extracted membrane fraction, as the CHAPSO concentration is raised stepwise to 1.0% (Table I). The amounts of γ-secretase activity that are solubilized by 1.0% and 2.0% CHAPSO are comparable. There are corresponding decreases in pellet-associated γ-secretase activity as a result of the CHAPSO extraction.

TABLE I
Extraction of γ-secretase activity with CHAPSO detergent

CHAPSO concentration

| | | | | | |
|---|---|---|---|---|---|
| γ-secretase | 0% | 0.25% | 0.50% | 1.00% | 2.0% |
| preparation | | | | | |
| Solubilized | <1 | 3 | 35 | 49 | 50 |
| Membranes | >99 | 97 | 65 | 51 | 50 |

Values show the % of total γ-secretase activity in each fraction (Solubilized versus Membranes) at each CHAPSO concentration.

Standard detergent solubilization of HeLa cell membranes (protein concentration, 2.5 mg/ml in buffer A) involved treatment with 1% CHAPSO for 60 minutes at 4° C. and centrifugation at 100,000×g for 60 minutes. The ensuing supernatant solution provides “solubilized γ-secretase”. Approximately 50% of the γ-secretase activity in the HeLa cell membranes is solubilized by this CHAPSO extraction protocol. The total recovery of γ-secretase activity in the “solubilized γ-secretase” and residual pellet is approximately 50% greater than that observed with the intact membranes (data not shown).

### Example 4

### In Vitro γ-Secretase Assay

In vitro assays measuring γ-secretase activity were performed using cell membranes or as “solubilized γ-secretase”. In one reaction, SEQ. ID. NO. 9 substrate (1.7 μM) was incubated with cell membranes (0.5 mg/ml) in presence of detergent in buffer B (50 mM PIPES, pH 7.0, 5 mM MgCl₂, 5 mM CaCl₂, 150 mM KCl) at 37° C. In another reaction, supernatant solution from CHAPSO-extracted HeLa cell membranes (“solubilized γ-secretase”) was incubated with SEQ. ID. NO. 9 substrate at 37° C. in the presence of detergent in buffer B. Generally, 0.25% CHAPSO was provided as the detergent. The reactions were stopped by adding RIPA (150 mM NaCl, 1.0% NP-40, 0.5% DOC, 0.1% SDS, 50 mM Tris HCl, pH 8.0).

The samples were centrifuged and the supernatant solutions were assayed for the Aβ peptides by ECL. The Aβ40- and Aβ42-related products from γ-secretase-mediated processing of SEQ. ID. NO. 9 substrate possess a Met at the N-terminus and are thus defined as M-Aβ40 and M-Aβ42, respectively.

### Example 5

### Inhibition Studies

Inhibition studies was performed to demonstrate that the γ-secretase activity in “solubilized γ-secretase” is catalyzed by the bona fide APP processing enzyme in cells and is not simply due to a spurious proteolytic activity. The studies examined the effects of L-685,458 on cleavage at the γ-secretase scissile bond of the substrates in both the cellular and in vitro assays.

L-685,458 is a putative γ-secretase inhibitor having the following structure:

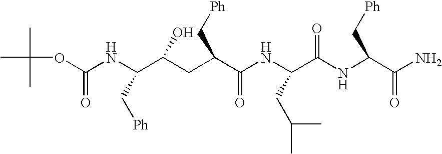

The effect of a γ-secretase inhibitor on γ-secretase activity was measured using Chinese hamster ovary fibroblasts that stably express APP695 (CHO/APP695, provided by Dr. S. Sisodia (University of Chicago, Chicago, Ill.)). CHO/APP695 were grown in 90% DMEM, 10% fetal bovine serum, 2 mM glutamine, 100 μg/ml each of penicillin and streptomycin, and 0.2 mg/ml G418. CHO/APP695 cells were seeded in 96-well dishes at 2×10⁴ cells/well. Aβ-peptide formation was detected by either ECL or using radiolabeling.

For ECL detection, the media was replenished the next day with or without L-685,458. The Aβ(x-40) and Aβ(x-42) levels in the 24 hour conditioned media (CM) were measured by ECL.

For the radiolabeling experiments, CHO/APP695 cells (in 100 mm dishes) were grown to 70% confluency in complete media. Afterwards, these cells were cultured ±1 μM L-685,458 for 24 hours, switched to Met-free medium (+/−1 μM L-685,458) for 20 minutes, pulsed with 1 mCi/ml of S³⁵-Met (Amersham Life Sciences, Inc, Arlington Heights, Ill.) in Met-free medium (+/−1 μM L-685,458) for 2 hours, and subsequently chased in complete medium (+/−1 μM L-685,458) for 45 minutes. The cells were washed with Hanks balanced salt solution and lysed with 1 ml of RIPA. The insoluble residue was removed by centrifugation. The 4G8 antibody (final concentration, 2 μg/ml) was added to the supernatant fractions. The samples were gently rotated overnight at 4° C. Protein G-agarose beads (Pharmacia Biotech, Piscataway, N.J.) were added the next day, the samples were rotated for an additional 2 hours and then centrifuged. The protein G-agarose beads from the 4G8 immunoprecipitation step were washed 4 times with RIPA. An equal volume of SDS sample buffer (Novex, San Diego, Calif.) was added and the samples were boiled for 5 minutes and fractionated by SDS-polyacrylamide gel electrophoresis (SDS-PAGE) using 16% polyacrylamide-Tricine gels (Novex). ¹⁴C-labeled protein markers (Amersham Life Sciences) were co-electrophoresed. The gel was dried and exposed to X-Omatic film (Kodak, Rochester, N.Y.) for 24-72 hours.

Treatment of CHO/APP695 cells with L-685,458 results in the intracellular accumulation of APP immunoreactive fragments that co-migrate with α-CTF and β-CTF (data not shown)—a result that is consistent with γ-secretase inhibition. Moreover, L-685,458 blocked Aβ40 and Aβ42 secretion from CHO/APP695 cells in a dose-dependent manner with IC₅₀ values for suppression of Aβ40 and Aβ42 secretion of 130 and 200 nM, respectively.

Similarly, L-685,458 inhibits “solubilized γ-secretase” mediated processing of SEQ. ID. NO. 9 substrate that results in the generation of the Aβ40- and Aβ42-related products. The IC₅₀ values for inhibition of the Aβ40 and Aβ42 cleavage events in the in vitro assay are both approximately 1 nM. All of the apparent “solubilized γ-secretase” activity is inhibited by L-685,458.

### Example 6

### Characterization of Product Formation

Incubation of HeLa cell membranes or “solubilized γ-secretase” with SEQ. ID. NO. 9 substrate generates M-Aβ42 and M-Aβ40. The Aβ42/Aβ40 cleavage ratios, approximately 0.1, with both intact membranes and “solubilized γ-secretase” are similar to the corresponding ratio that has been reported for Aβ peptides secreted into the CM of cultured cells that are overexpressing APP (Asami-Odaka et al., 1995. Biochemistry 34, 10272-10278).

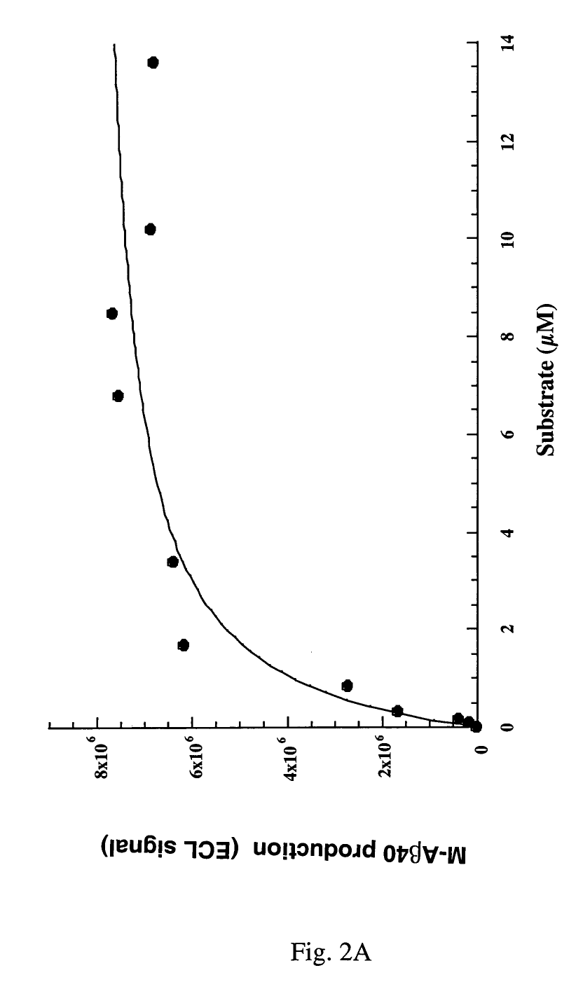

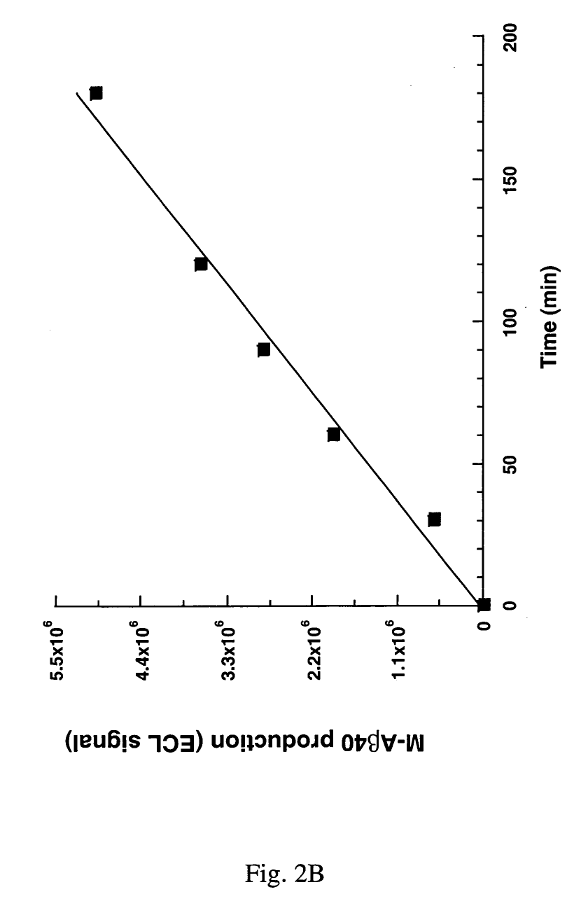

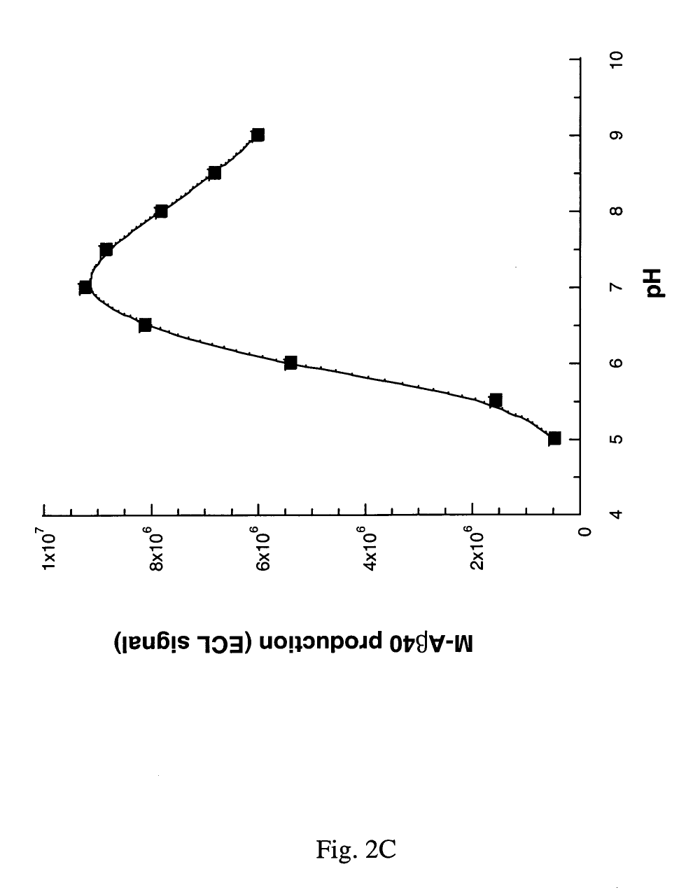

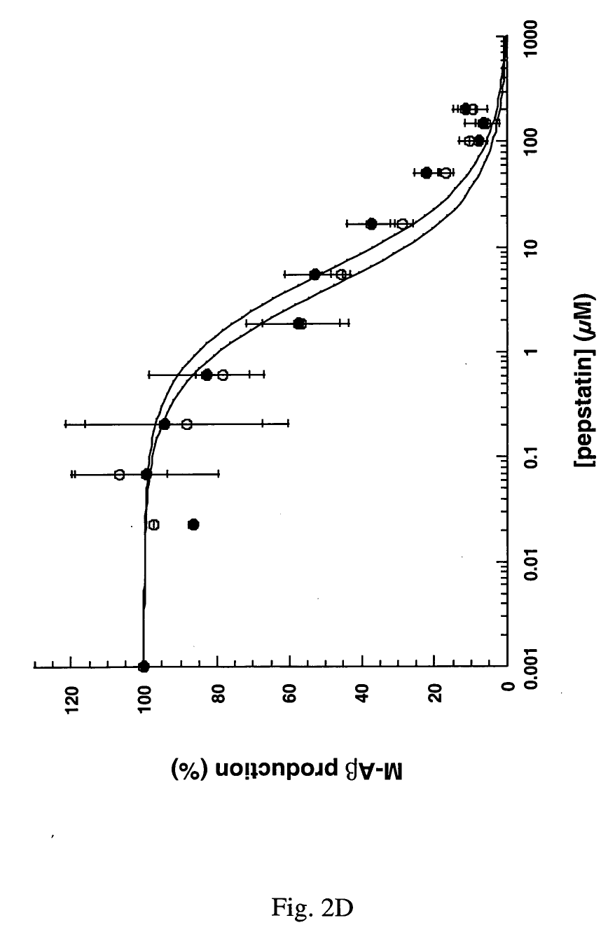

The apparent Kₘ value of “solubilized γ-secretase” for SEQ. ID. NO. 9 substrate (assaying for Aβ40 cleavage) is approximately 1 μM (FIG. 2A). Under the experimental conditions employed, the time dependence of product formation is linear over a 3 hour interval (FIG. 2B). The production of the M-Aβ40 and M-Aβ42 are both blocked by pepstatin, a classical inhibitor of aspartyl class proteases (FIG. 2D). The IC₅₀ values of pepstatin for inhibiting the generation of the Aβ40 and Aβ42 termini are 4.0 and 5.9 μM, respectively.

### Example 7

### pH Dependency

The pH dependence of in vitro γ-secretase activity was determined using pH-adjusted 50 mM MES and 50 mM PIPES for the pH 5.0-6.5 and pH 6.5-9.0 ranges, respectively. The pH dependence of “solubilized γ-secretase” activity showed a pH optimum of 7.0 (FIG. 2C).

### Example 8

### Detergent Effect

SEQ. ID. NO. 9 substrate (1.7 μM) was incubated with cell membranes (0.5 mg/ml) in presence of CHAPSO, CHAPS or Triton X-100 (0, 0.125, 0.25, 0.5, or 1%) in buffer B at 37° C. The results are shown in Table II.

TABLE II
M-Aβ 40 production (ECL signal × 10⁻⁶)

| [Detergent] | | | |
|---|---|---|---|
| (%) | CHAPSO | CHAPS | Triton X-100 |
| 0 | 0.03 | 0.03 | 0.03 |
| 0.125 | 2.2 | 0.03 | 0.02 |
| 0.25 | 8.74 | 3.21 | 0.02 |
| 0.5 | 1.22 | 0.27 | 0.02 |
| 0.75 | 0.07 | 0.04 | 0.02 |
| 1 | 0.03 | 0.03 | 0.02 |

Simple addition of SEQ. ID. NO. 9 substrate to membranes prepared from HeLa cells does not lead to substrate cleavage and generation of M-Aβ40. Inclusion of CHAPSO or CHAPS during the incubation of HeLa cell membranes and SEQ. ID. NO. 9 substrate promotes cleavage at the γ-secretase scissile bond. Triton X-100 fails to potentiate in vitro γ-secretase activity. Optimal activity is observed in the presence of 0.25% CHAPSO. Higher concentrations of CHAPSO lead to a progressive decline in γ-secretase activity. Membranes prepared from human embryonic kidney (HEK293) cells or Chinese hamster ovary (CHO) cells also process SEQ. ID. NO. 9 substrate in the presence of 0.25% CHAPSO to generate M-Aβ40 (data not shown).

Other embodiments are within the following claims. While several embodiments have been shown and described, various modifications may be made without departing from the spirit and scope of the present invention.

1-19. (canceled)

20. A method for assaying γ-secretase activity comprising:
a. combining together a γ-secretase substrate and a preparation having γ-secretase activity, under reaction conditions allowing for γ-secretase activity to occur; and
b. measuring γ-secretase activity,
wherein the γ-secretase substrate comprises a hydrophilic peptide moiety covalently joined to the carboxyl terminus of a β-CTF domain, and
wherein the reaction conditions allowing for γ-secretase activity comprises an effective amount of a zwitterionic detergent.

21. The method of claim 20 wherein the zwitterionic detergent is selected from the group consisting of CHAPSO and CHAPS.

22. The method of claim 21 wherein the effective amount is about 0.1%-0.5%.

23. The method of claim 22 wherein said measuring comprises the step of detecting formation of an Aβ peptide.

24. The method of claim 23 wherein said measuring comprises the use of a binding substance specific for an epitope at the carboxyl terminus of the Aβ peptide.

25. The method of claim 24 wherein said binding substance is selected from the group consisting of antibodies and radioactive, electrochemiluminescent or fluorescent labels.

26. A method for assaying the ability of a compound to affect γ-secretase activity comprising:
a. combining together a γ-secretase substrate, a compound and a preparation having γ-secretase activity, under reaction conditions allowing for γ-secretase activity to occur; and
b. measuring γ-secretase activity,
wherein the γ-secretase substrate comprises a hydrophilic peptide moiety covalently joined to the carboxyl terminus of a β-CTF domain, and
wherein the reaction conditions allowing for γ-secretase activity comprises an effective amount of a zwitterionic detergent.

27. The method of claim 26 wherein the zwitterionic detergent is selected from the group consisting of CHAPSO and CHAPS.

28. The method of claim 27 wherein the effective amount is about 0.1%-0.5%.

29. The method of claim 28 wherein said measuring comprises the step of detecting formation of an Aβ peptide.

30. The method of claim 29 wherein said measuring comprises the use of a binding substance specific for an epitope at the carboxyl terminus of the Aβ peptide.

31. The method of claim 30 wherein said binding substance is selected from the group consisting of antibodies and radioactive, electrochemiluminescent or fluorescent labels.

32. A method for assaying for γ-secretase activity comprising:
a. providing a reaction system including a solubilized γ-secretase, a zwitterionic detergent and a γ-secretase substrate, wherein the γ-secretase substrate comprises a hydrophilic peptide moiety covalently joined to the carboxyl terminus of a β-CTF domain, under reaction conditions which permit γ-secretase cleavage of the γ-secretase substrate into γ-secretase cleavage products; and
b. detecting the amount of at least one γ-secretase cleavage product,
wherein the reaction conditions allowing for γ-secretase activity comprises an effective amount of a zwitterionic detergent.

33. The method of claim 32 wherein the zwitterionic detergent is selected from the group consisting of CHAPSO and CHAPS.

34. The method of claim 33 wherein the effective amount is about 0.1%-O.5%.

35. The method of claim 34 wherein said measuring comprises the step of detecting formation of an Aβ peptide.

36. The method of claim 35 wherein said measuring comprises the use of a binding substance specific for an epitope at the carboxyl terminus of the Aβ peptide.

37. The method of claim 36 wherein said binding substance is selected from the group consisting of antibodies and radioactive, electrochemiluminescent or fluorescent labels.
